(19) United States
(12) Patent Application Publication
(10) Pub. No.: US 2023/0130225 A1
(43) Pub. Date: Apr. 27, 2023

(54) METHOD OF TREATING AMYOTROPHIC LATERAL SCLEROSIS WITH MYELOPEROXIDASE INHIBITOR

(71) Applicant: Biohaven Therapeutics Ltd., New Haven, CT (US)

(72) Inventors: Irfan QURESHI, Hackensack, NJ (US); Marianne FROST, North Haven, CT (US); Mary K. DONOHUE, Wallingford, CT (US)

(21) Appl. No.: 17/907,784

(22) PCT Filed: Mar. 5, 2021
(86) PCT No.: PCT/US2021/020979

§ 371 Date: Aug. 29, 2022

Related U.S. Application Data
(60) Provisional application No. 62/985,802, filed on Mar. 5, 2020.

(51) Int. Cl. A61K 31/519; A61K 31/428; A61P 25/28

(52) U.S. Cl. CPC A61K 31/519; A61K 31/428; A61P 25/28

(57) ABSTRACT

Provided is a method for treating amyotrophic lateral sclerosis, including administering to a subject in need of such treatment an effective amount of a myeloperoxidase inhibitor or a pharmaceutically acceptable salt thereof.

### CROSS-REFERENCE TO RELATED APPLICATIONS

This application claims priority to U.S. Provisional Application No. 62/985,802 filed Mar. 5, 2020 and all the benefits accruing therefrom under 35 U.S.C. § 119, the content of which is incorporated herein in its entirety by reference.

### FIELD OF THE INVENTION

The present invention relates methods of treating amyotrophic lateral sclerosis (ALS) with therapeutically active compositions. Specifically, the present invention relates to methods of treating ALS by administering a pharmaceutical composition containing a myeloperoxidase (MPO) inhibitor.

### BACKGROUND OF THE INVENTION

Amyotrophic lateral sclerosis (ALS) is a paralyzing disorder that results in patient death within a few years after onset. Clinical symptoms primarily result from progressive motoneuron degeneration in the spinal cord and brain stem that lead to decline in cognitive performance. The disease affects healthy individuals in the midst of their life, in many cases without any family history. Although more males than females seem to be affected before the age of 60 years, both genders are similarly affected at older age. With the ageing population, increasingly more individuals suffer ALS, making it the third most common neurodegenerative disorder. Many ALS patients first notice muscle weakness in their limbs (“limb-onset” ALS). In ~25% of ALS patients, motoneurons first degenerate in the motor nuclei of the brain stem (“bulbar-onset” ALS), causing dysarthria, dysphagia, and respiratory problems. Bulbar-onset ALS patients generally exhibit a faster and more aggressive disease progression than do limb-onset patients, but the latter eventually develop bulbar symptoms as well.

The precise cause of motoneuron degeneration in most cases remains unknown. It has been determined that SOD-1 mutations cause motoneuron degeneration in humans and, when overexpressed, also in transgenic mice. SOD-1^{G93A} mice have become the standard animal model to assess the therapeutic potential of novel drug candidates.

No approved, effective cure is available yet for ALS. Riluzole and edaravone are the only approved small-molecule drug in some but not all countries. However, riluzole has a marginal benefit on survival, is costly, not free of side-effects and, importantly, ineffective on bulbar symptoms. Apart from the small molecules, clinical applicability of gene therapy for ALS remains to be established and concerns about its irreversible nature, risk for adverse chromosomal effects, poor control of transgene expression, and large production needs still remain to be overcome.

There is therefore a large unmet need for medications that can be used for the ALS treatment.

### SUMMARY OF THE INVENTION

The present invention is directed to the treatment of amyotrophic lateral sclerosis (ALS) by administering a pharmaceutical composition containing a myeloperoxidase (MPO) inhibitor.

In an embodiment, provided is a method for treating amyotrophic lateral sclerosis, comprising administering to a subject in need of such treatment an effective amount of 1-(2-isopropoxyethyl)-2-thioxo-1,2,3,5-tetrahydro-pyrrolo[3,2-d]pyrimidin-4-one or a pharmaceutically acceptable salt thereof.

The treating may include slowing progression of amyotrophic lateral sclerosis, reducing intensity of symptoms associated with amyotrophic lateral sclerosis, reducing onset of symptoms associated with amyotrophic lateral sclerosis, reducing weight loss associated with amyotrophic lateral sclerosis, reversing weight loss associated with amyotrophic lateral sclerosis, delaying mortality associated with amyotrophic lateral sclerosis, or combinations thereof.

The symptoms associated with amyotrophic lateral sclerosis may be selected from the group consisting of fine motor function, gross motor function, bulbar function, respiratory function, cognitive function, and a combinations thereof.

The symptoms associated with amyotrophic lateral sclerosis may be selected from the group consisting of walking, speech, eating, swallowing, writing, climbing stairs, cutting food, turning in bed, salivation, dressing, maintaining hygiene, breathing, dyspnea, orthopnea, respiratory insufficiency, and combinations thereof.

In an embodiment, the effective amount may be about 150 mg or more per day. In another embodiment, the effective amount may be about 300 mg or more per day. In another embodiment, the effective amount is about 600 mg or more per day. In another embodiment, the effective amount may be about 900 mg per day. In another embodiment, the effective amount may be about 1200 mg per day.

In an embodiment, the administering may include administering a dose equal to about half of the daily dose two times per day. In another embodiment, the administering may include administering a dose equal to about half of a daily dose every 12 hours. In another embodiment, the administering may include administering a dose equal to about one quarter of a daily dose four times per day. In another embodiment, the administering comprises administering about 600 mg two times per day. In another embodiment, the administering comprises administering about 300 mg four times per day.

In an embodiment, the method may be carried out for a time period selected from the group consisting of at least about 12 weeks, at least about 6 months, at least about 1 year, at least about 2 years, at least about 3 years, at least about 4 years, at least about 5 years, at least about 10 years, and until the patient dies. In another embodiment, the method may be carried out at least daily for an indefinite amount of time.

The method may further include administering one or more other amyotrophic lateral sclerosis treatments simultaneously or concurrently with administering 1-(2-isopropoxyethyl)-2-thioxo-1,2,3,5-tetrahydro-pyrrolo[3,2-d]pyrimidin-4-one or a pharmaceutically acceptable salt thereof. In an embodiment, the one or more other amyotrophic lateral sclerosis treatment may include riluzole. For example, the one or more other amyotrophic lateral sclerosis treatment may include a sublingual formulation of riluzole. In another embodiment, the one or more other amyotrophic lateral sclerosis treatment may include a prodrug of riluzole. For example, the prodrug of riluzole may be troriluzole.

In an embodiment, the patient may have begun exhibiting symptoms of amyotrophic lateral sclerosis less than about two years before beginning administering 1-(2-isopropoxyethyl)-2-thioxo-1,2,3,5-tetrahydro-pyrrolo[3,2-d]pyrimidin-4-one or a pharmaceutically acceptable salt thereof. In another embodiment, the patient may have begun exhibiting symptoms of amyotrophic lateral sclerosis at least greater than about two years before beginning administering 1-(2-isopropoxyethyl)-2-thioxo-1,2,3,5-tetrahydro-pyrrolo[3,2-d]pyrimidin-4-one or a pharmaceutically acceptable salt thereof.

In an embodiment, the patient may exhibit a greater than 20% improvement in ALS Functional Rating Scale, Revised (ALSFRS-R) score when compared to baseline. In another embodiment, the patient may exhibit a greater than 30% improvement in ALS Functional Rating Scale, Revised (ALSFRS-R) score when compared to baseline.

The improvement may be apparent in a time period selected from the group consisting of less than about 9 months, less than about 6 months, less than about 3 months, and less than about 1 month.

The administering 1-(2-isopropoxyethyl)-2-thioxo-1,2,3,5-tetrahydro-pyrrolo[3,2-d]pyrimidin-4-one or a pharmaceutically acceptable salt thereof may result in slowing of a rate of fine motor function loss in the patient.

The method may further include administering a daily dose of greater than an effective amount for a period of time before administering an effective amount of 1-(2-isopropoxyethyl)-2-thioxo-1,2,3,5-tetrahydro-pyrrolo[3,2-d]pyrimidin-4-one or a pharmaceutically acceptable salt thereof. In an embodiment, the effective amount may be greater than 600 mg. In another embodiment, the effective amount may be greater than 900 mg. In another embodiment, the effective amount is greater than 1200 mg.

In an embodiment, the period of time before administering a greater than effective amount may be from about 1 weeks to about 12 weeks. In another embodiment, the period of time before administering a greater than effective amount may be from about 2 weeks to about 6 weeks. In another embodiment, the administering a greater than effective amount of 1-(2-isopropoxyethyl)-2-thioxo-1,2,3,5-tetrahydro-pyrrolo[3,2-d]pyrimidin-4-one or a pharmaceutically acceptable salt thereof may be carried out indefinitely.

An effective amount of 1-(2-isopropoxyethyl)-2-thioxo-1,2,3,5-tetrahydro-pyrrolo[3,2-d]pyrimidin-4-one or a pharmaceutically acceptable salt thereof may be administered in an initial dose and every administration thereafter.

The effective amount may include a stable daily dose. In an embodiment, the stable daily dose may include about 1200 mg of 1-(2-isopropoxyethyl)-2-thioxo-1,2,3,5-tetrahydro-pyrrolo[3,2-d]pyrimidin-4-one or a pharmaceutically acceptable salt thereof. In another embodiment, the stable daily dose may include 1 to 5 unit doses per day. Each unit dose may be a solid unit dose.

In an embodiment, the administering may include administering one unit dose two times per day wherein each unit dose is equal to about half of the stable daily dose. In another embodiment, the administering may include administering one unit dose once every 12 hours wherein each unit dose is equal to about half of the stable daily dose. In another embodiment, the administering may include administering one unit dose four times per day wherein each unit dose is equal to about one quarter of the stable daily dose.

In an embodiment, the administering may include administering two unit doses wherein each unit dose is about 600 mg two times per day. In another embodiment, the administering may include administering four unit doses wherein each unit dose is about 300 mg four times per day.

In an embodiment, the administering a stable daily dose of 1-(2-isopropoxyethyl)-2-thioxo-1,2,3,5-tetrahydro-pyrrolo[3,2-d]pyrimidin-4-one or a pharmaceutically acceptable salt thereof may be carried out for at least about 12 weeks. In another embodiment, the administering a stable daily dose of 1-(2-isopropoxyethyl)-2-thioxo-1,2,3,5-tetrahydro-pyrrolo[3,2-d]pyrimidin-4-one or a pharmaceutically acceptable salt thereof may be carried out for an indefinite amount of time.

In an embodiment, the stable daily dose may be consistent throughout a treatment regimen. In another embodiment, an initial daily dose may be equal to each daily dose thereafter. In an embodiment, there is no titration before administering the stable daily dose.

In an embodiment, the method may further include monitoring the patient. For example, the method may include monitoring the patient for neutropenia. In another embodiment, the method may further include monitoring ALSFRS-R score for the patient. In another embodiment, the method may further include monitoring the patient’s fine motor function, gross motor function, bulbar function, respiratory function, or a combinations thereof. In another embodiment, the method may further include monitoring behaviors selected from the group consisting of swallowing, handwriting, speech, ability to walk, ability to climb stairs, ability to dress, ability to maintain hygiene, and combinations thereof. In an embodiment, the method may further include scheduling a doctor visit every 6 months for at least 12 months.

In an embodiment, the patient may be predisposed to amyotrophic lateral sclerosis and is not exhibiting symptoms of amyotrophic lateral sclerosis.

The method may further include administering 1-(2-isopropoxyethyl)-2-thioxo-1,2,3,5-tetrahydro-pyrrolo[3,2-d]pyrimidin-4-one or a pharmaceutically acceptable salt thereof to family members of the patient.

The treating may include administering 1-(2-isopropoxyethyl)-2-thioxo-1,2,3,5-tetrahydro-pyrrolo[3,2-d]pyrimidin-4-one or a pharmaceutically acceptable salt thereof to a patient not exhibiting symptoms of amyotrophic lateral sclerosis.

The treating may include administering 1-(2-isopropoxyethyl)-2-thioxo-1,2,3,5-tetrahydro-pyrrolo[3,2-d]pyrimidin-4-one or a pharmaceutically acceptable salt thereof to a patient that is predisposed to amyotrophic lateral sclerosis.

### DETAILED DESCRIPTION OF THE INVENTION

The following detailed description is provided to aid those skilled in the art in practicing the present invention. Those of ordinary skill in the art may make modifications and variations in the embodiments described herein without departing from the spirit or scope of the present disclosure. Unless otherwise defined, all technical and scientific terms used herein have the same meaning as commonly understood by one of ordinary skill in the art to which this disclosure belongs. The terminology used in the description is for describing particular embodiments only and is not intended to be limiting.

As used in this application, except as otherwise expressly provided herein, each of the following terms shall have the meaning set forth below. Additional definitions are set forth throughout the application. In instances where a term is not specifically defined herein, that term is given an art-recognized meaning by those of ordinary skill applying that term in context to its use in describing the present invention.

The articles “a” and “an” refer to one or to more than one (i.e., to at least one) of the grammatical object of the article unless the context clearly indicates otherwise. By way of example, “an element” means one element or more than one element.

The term “about” refers to a value or composition that is within an acceptable error range for the particular value or composition as determined by one of ordinary skill in the art, which will depend in part on how the value or composition is measured or determined, i.e., the limitations of the measurement system. For example, “about” can mean within 1 or more than 1 standard deviation per the practice in the art. Alternatively, “about” can mean a range of up to 1%, 5%, 10% or 20% (i.e., ±10% or ±20%) depending on the context of the application. For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” should be assumed to be within an acceptable error range for that particular value or composition.

The term “administering” refers to the physical introduction of a composition comprising a therapeutic agent to a subject, using any of the various methods and delivery systems known to those skilled in the art. Administering can also be performed, for example, once, a plurality of times, and/or over one or more extended periods and can be a therapeutically effective dose or a subtherapeutic dose.

The term “Cₘₐₓ” refers to a maximum concentration of a drug in blood, serum, a specified compartment or test area of a subject between administration of a first dose and administration of a second dose. The term Cₘₐₓ could also refer to dose normalized ratios, if specified.

The term “dosing interval,” refers to the amount of time that elapses between multiple doses of a formulation disclosed herein being administered to a subject. Dosing interval can thus be indicated as ranges.

The term “dosing frequency” refers to the frequency of administering doses of a formulation disclosed herein in a given time. Dosing frequency can be indicated as the number of doses per a given time, e.g., once a week or once in two weeks.

The terms “in combination with” and “in conjunction with” refer to administration of one treatment modality in addition to another treatment modality. As such, “in combination with” or “in conjunction with” refers to administration of one treatment modality before, during, or after administration of the other treatment modality to the subject.

The term “pharmaceutically acceptable salt” refers to a salt form of one or more of the compounds described herein which are typically presented to increase the solubility of the compound in the gastric or gastroenteric juices of the patient’s gastrointestinal tract in order to promote dissolution and the bioavailability of the compounds. Pharmaceutically acceptable salts include those derived from pharmaceutically acceptable inorganic or organic bases and acids, where applicable. Suitable salts include, for example, those derived from alkali metals such as potassium and sodium, alkaline earth metals such as calcium, magnesium and ammonium salts, among numerous other acids and bases well known in the pharmaceutical art.

The terms “subject” and “patient” refer any human or nonhuman animal. The term “nonhuman animal” includes, but is not limited to, vertebrates such as nonhuman primates, sheep, dogs, and rodents such as mice, rats and guinea pigs. In some embodiments, the subject is a human. The terms, “subject” and “patient” are used interchangeably herein.

The terms “effective amount”, “therapeutically effective amount”, “therapeutically effective dosage” and “therapeutically effective dose” of an agent (also sometimes referred to herein as a “drug”) refers to any amount of the agent that, when used alone or in combination with another agent, protects a subject against the onset of a disease or promotes disease regression evidenced by a decrease in severity of disease symptoms, an increase in frequency and duration of disease symptom-free periods, or relief from impairment or disability due to the disease affliction. The therapeutically effective amount of an agent can be evaluated using a variety of methods known to the skilled practitioner, such as in human subjects during clinical trials, in animal model systems predictive of efficacy in humans, or by assaying the activity of the agent in in vitro assays.

The term “treatment” refers to any treatment of a condition or disease in a subject and may include: (i) preventing the disease or condition from occurring in the subject which may be predisposed to the disease but has not yet been diagnosed as having it; (ii) inhibiting the disease or condition, i.e., arresting its development; relieving the disease or condition, i.e., causing regression of the condition; or (iii) ameliorating or relieving the conditions caused by the disease, i.e., symptoms of the disease. Treatment could be used in combination with other standard therapies or alone. Treatment or “therapy” of a subject also includes any type of intervention or process performed on, or the administration of an agent to, the subject with the objective of reversing, alleviating, ameliorating, inhibiting, slowing down or preventing the onset, progression, development, severity or recurrence of a symptom, complication or condition, or biochemical indicia associated with a disease.

With respect to the disease, “treatment” is an approach for obtaining beneficial or desired clinical results. For purposes of this invention, beneficial or desired clinical results include, but are not limited to, one or more of the following: improvement in any aspect of a major symptom including lessening severity, alleviation of major symptom intensity, and other associated symptoms, reducing frequency of recurrence, increasing the quality of life of those suffering from the symptom, and decreasing dose of other medications required to treat the symptom.

The starting materials useful for making the pharmaceutical compositions of the present invention are readily commercially available or can be prepared by those skilled in the art.

In an embodiment, a method of treating ALS is provided. The method includes administering to a patient having ALS an effective amount of an MPO inhibitor or a pharmaceutically acceptable salt thereof. The MPO inhibitor may be 1-(2-isopropoxyethyl)-2-thioxo-1,2,3,5-tetrahydro-pyrrolo[3,2-d]pyrimidin-4-one (verdiperstat) or a pharmaceutically acceptable salt thereof:

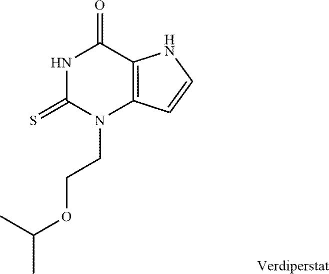

The method of treating according to embodiments of the present invention may slow progression of amyotrophic lateral sclerosis, reduce intensity of symptoms associated with amyotrophic lateral sclerosis, reduce onset of symptoms associated with amyotrophic lateral sclerosis, reduce weight loss associated with amyotrophic lateral sclerosis, reverse weight loss associated with amyotrophic lateral sclerosis, delay mortality associated with amyotrophic lateral sclerosis, or may result in a combination of the above effects.

The symptoms associated with amyotrophic lateral sclerosis may include fine motor function, gross motor function, balbar function, respiratory function, or a combinations thereof.

The symptoms associated with amyotrophic lateral sclerosis may include walking, speech, eating, swallowing, writing, climbing stairs, cutting food, turning in bed, salivation, dressing, maintaining hygiene, breathing, dyspnea, orthopnea, respiratory insufficiency, or a combination thereof.

In an embodiment, the effective amount of verdiperstat may be from about 50 mg to about 3000 mg, for example, from about 100 mg to about 2000 mg, or from about 600 mg to about 1200 mg. For example, the effective amount of verdiperstat may be about 100 mg or more per day, about 150 mg or more per day, about 200 mg or more per day, about 250 mg or more per day, about 300 mg or more per day, about 350 mg or more per day, about 400 mg or more per day, about 450 mg or more per day, about 500 mg or more per day, about 550 mg or more per day, about 600 mg or more per day, about 650 mg or more per day, about 700 mg or more per day, about 750 mg or more per day, about 800 mg or more per day, about 850 mg or more per day, about 900 mg or more per day, about 950 mg or more per day, about 1000 mg or more per day, about 1100 mg or more per day, about 1200 mg or more per day, about 1300 mg or more per day, about 1400 mg or more per day, about 1500 mg or more per day, about 1600 mg or more per day, about 1700 mg or more per day, about 1800 mg or more per day, about 1900 mg or more per day, or about 2000 mg or more per day. For example, the effective amount of verdiperstat may be about 300 mg or more per day, about 400 mg or more per day, about 500 mg or more per day, about 600 mg or more per day, about 700 mg or more per day, about 800 mg or more per day, about 900 mg or more per day, about 1000 mg or more per day, about 1100 mg or more per day, or about 1200 mg or more per day.

In an embodiment, the effective amount of verdiperstat may be about 600 mg or more per day. In another embodiment, the effective amount of verdiperstat may be about 900 mg per day. In another embodiment, the effective amount of verdiperstat may be about 1200 mg per day. The effective amount of verdiperstat may be administered once per day (QD dosing), two times per day (BID dosing), three times per day, or four times per day.

In an embodiment, a dose equal to about half of the daily dose may be administered two times per day. In another embodiment, a dose equal to about half of a daily dose may be administered every 12 hours. In another embodiment, a dose equal to about one quarter of a daily dose may be administered four times per day. In another embodiment, about 600 mg of verdiperstat may be administered two times per day. In another embodiment, the administering about 300 mg of verdiperstat may be administered four times per day.

In an embodiment, the treatment may be carried out for a time period selected from the group consisting of at least about 12 weeks, at least about 24 weeks, at least about 6 months, at least about 1 year, at least about 2 years, at least about 3 years, at least about 4 years, at least about 5 years, at least about 10 years, and until the patient dies. In another embodiment, the treatment may be carried out at least daily for an indefinite amount of time.

One or more other amyotrophic lateral sclerosis treatments may be administered simultaneously or concurrently with administering 1-(2-isopropoxyethyl)-2-thioxo-1,2,3,5-tetrahydro-pyrrolo[3,2-d]pyrimidin-4-one or a pharmaceutically acceptable salt thereof. In an embodiment, the one or more other amyotrophic lateral sclerosis treatment may include riluzole. The one or more other amyotrophic lateral sclerosis treatment may include a sublingual formulation of riluzole, which may be a lyophilized, tableted sublingual formulation of riluzole. In another embodiment, the one or more other amyotrophic lateral sclerosis treatment may include a prodrug of riluzole. The prodrug of riluzole may be troriluzole.

The patient may have begun exhibiting symptoms of amyotrophic lateral sclerosis before beginning administering 1-(2-isopropoxyethyl)-2-thioxo-1,2,3,5-tetrahydro-pyrrolo[3,2-d]pyrimidin-4-one or a pharmaceutically acceptable salt thereof. In an embodiment, the patient may begin administering symptoms of amyotrophic lateral sclerosis less than about two years before beginning administering 1-(2-isopropoxyethyl)-2-thioxo-1,2,3,5-tetrahydro-pyrrolo[3,2-d]pyrimidin-4-one or a pharmaceutically acceptable salt thereof. In another embodiment, the patient may have begun exhibiting symptoms of amyotrophic lateral sclerosis at least greater than about two years before beginning administering 1-(2-isopropoxyethyl)-2-thioxo-1,2,3,5-tetrahydro-pyrrolo[3,2-d]pyrimidin-4-one or a pharmaceutically acceptable salt thereof.

The patient may exhibit a greater than 10% improvement in ALS Functional Rating Scale, Revised (ALSFRS-R) score when compared to baseline. For example, the patient may exhibit a greater than 15%, a greater than 20%, a greater than 25%, a greater than 30%, a greater than 35%, a greater than 40%, a greater than 45%, a greater than 50%, a greater than 55%, a greater than 60%, a greater than 65%, a greater than 70%, a greater than 75%, a greater than 80%, a greater than 85%, a greater than 90%, or a greater than 95% improvement in ALS Functional Rating Scale, Revised (ALSFRS-R) score when compared to baseline. In an embodiment, the patient may exhibit a greater than 20% improvement in ALS Functional Rating Scale, Revised (ALSFRS-R) score when compared to baseline. In another embodiment, the patient may exhibit a greater than 30% improvement in ALS Functional Rating Scale, Revised (ALSFRS-R) score when compared to baseline.

The improvement may be apparent in a time period selected from the group consisting of less than about 9 months, less than about 8 months, less than about 7 months, less than about 6 months, less than about 5 months, less than about 4 months, less than about 3 months, less than about 2 months, and less than about 1 month. For example, the improvement may be apparent in a time period selected from the group consisting of less than about 9 months, less than about 6 months, less than about 3 months, and less than about 1 month.

The administering 1-(2-isopropoxyethyl)-2-thioxo-1,2,3,5-tetrahydro-pyrrolo[3,2-d]pyrimidin-4-one or a pharmaceutically acceptable salt thereof may result in slowing of a rate of fine motor function loss in the patient.

The method may further include administering a daily dose of greater than an effective amount for a period of time before administering an effective amount of 1-(2-isopropoxyethyl)-2-thioxo-1,2,3,5-tetrahydro-pyrrolo[3,2-d]pyrimidin-4-one or a pharmaceutically acceptable salt thereof. In an embodiment, the effective amount may be greater than 600 mg. In another embodiment, the effective amount may be greater than 900 mg. In another embodiment, the effective amount is greater than 1200 mg.

In an embodiment, the period of time before administering a greater than effective amount may be from about 1 weeks to about 12 weeks. In another embodiment, the period of time before administering a greater than effective amount may be from about 2 weeks to about 6 weeks. In another embodiment, the administering a greater than effective amount of 1-(2-isopropoxyethyl)-2-thioxo-1,2,3,5-tetrahydro-pyrrolo[3,2-d]pyrimidin-4-one or a pharmaceutically acceptable salt thereof may be carried out indefinitely.

An effective amount of 1-(2-isopropoxyethyl)-2-thioxo-1,2,3,5-tetrahydro-pyrrolo[3,2-d]pyrimidin-4-one or a pharmaceutically acceptable salt thereof may be administered in an initial dose and every administration thereafter.

The effective amount may include a stable daily dose. In an embodiment, the stable daily dose may include about 1200 mg of 1-(2-isopropoxyethyl)-2-thioxo-1,2,3,5-tetrahydro-pyrrolo[3,2-d]pyrimidin-4-one or a pharmaceutically acceptable salt thereof. In another embodiment, the stable daily dose may include 1 to 5 unit doses per day. Each unit dose may be a solid unit dose.

In an embodiment, the administering may include administering one unit dose two times per day wherein each unit dose is equal to about half of the stable daily dose. In another embodiment, the administering may include administering one unit dose once every 12 hours wherein each unit dose is equal to about half of the stable daily dose. In another embodiment, the administering may include administering one unit dose four times per day wherein each unit dose is equal to about one quarter of the stable daily dose.

In an embodiment, the administering may include administering two unit doses wherein each unit dose is about 600 mg two times per day. In another embodiment, the administering may include administering three unit doses wherein each unit dose is about 400 mg four times per day. In another embodiment, the administering may include administering four unit doses wherein each unit dose is about 300 mg four times per day.

In an embodiment, the administering a stable daily dose of 1-(2-isopropoxyethyl)-2-thioxo-1,2,3,5-tetrahydro-pyrrolo[3,2-d]pyrimidin-4-one or a pharmaceutically acceptable salt thereof may be carried out for at least about 12 weeks. In another embodiment, the administering a stable daily dose of 1-(2-isopropoxyethyl)-2-thioxo-1,2,3,5-tetrahydro-pyrrolo[3,2-d]pyrimidin-4-one or a pharmaceutically acceptable salt thereof may be carried out for an indefinite amount of time.

In an embodiment, the stable daily dose may be consistent throughout a treatment regimen. In another embodiment, an initial daily dose may be equal to each daily dose thereafter. In an embodiment, there is no titration before administering the stable daily dose.

In an embodiment, the method may further include monitoring the patient. For example, the method may include monitoring the patient for neutropenia. In another embodiment, the method may further include monitoring ALSFRS-R score for the patient. In another embodiment, the method may further include monitoring the patient’s fine motor function, gross motor function, bulbar function, respiratory function, or a combinations thereof. In another embodiment, the method may further include monitoring behaviors selected from the group consisting of swallowing, handwriting, speech, ability to walk, ability to climb stairs, ability to dress, ability to maintain hygiene, and combinations thereof. In an embodiment, the method mayfurther include scheduling a doctor visit every 6 months for at least 12 months.

In an embodiment, the patient may be predisposed to amyotrophic lateral sclerosis and is not exhibiting symptoms of amyotrophic lateral sclerosis.

The method may further include administering 1-(2-isopropoxyethyl)-2-thioxo-1,2,3,5-tetrahydro-pyrrolo[3,2-d]pyrimidin-4-one or a pharmaceutically acceptable salt thereof to family members of the patient.

The treating may include administering 1-(2-isopropoxyethyl)-2-thioxo-1,2,3,5-tetrahydro-pyrrolo[3,2-d]pyrimidin-4-one or a pharmaceutically acceptable salt thereof to a patient not exhibiting symptoms of amyotrophic lateral sclerosis.

The treating may include administering 1-(2-isopropoxyethyl)-2-thioxo-1,2,3,5-tetrahydro-pyrrolo[3,2-d]pyrimidin-4-one or a pharmaceutically acceptable salt thereof to a patient that is predisposed to amyotrophic lateral sclerosis.

### Introduction Regimen: Verdiperstat (BHV-3241)

### Verdiperstat Background Information

The invention encompasses a method of treating neurodegenerative diseases by administration of an MPO inhibitor, including amyotrophic lateral sclerosis (ALS). In an embodiment, the MPO inhibitor may be verdiperstat (also known as BHV-3241).

Verdiperstat is a first-in-class, potent, selective, brain-permeable, irreversible inhibitor of myeloperoxidase (MPO) enzyme. MPO is one of the most abundant enzymes in activated myeloid cells, including microglia [1]. It is a lysosomal enzyme that plays essential roles in immune surveillance and host defense. In disease, innate immune system activation leads to MPO-induced pathological oxidative stress and further inflammation that contribute to cellular injury [1]. Increasing evidence suggests MPO is involved in several neurodegenerative diseases [1, 2].

The proposed study is based on cumulative non-clinical, clinical, and neuroimaging information that implicate MPO activity in the onset and progression of neurodegenerative diseases. The present inventors believe that treatment with verdiperstat has the potential to slow neurodegeneration. The high unmet need for an effective treatment, together with the available data, provide a compelling rationale for the development of verdiperstat as a treatment for ALS.

### Verdiperstat Rationale

### Oxidative Stress

Based on extensive supportive evidence from human ALS and animal models, oxidative stress is thought to promote neurodegeneration in ALS and other disorders [4]. MPO catalyzes generation cytotoxic oxidizing and nitrosylating compounds, e.g., hypochlorous acid and peroxynitrite [5, 6] which are strongly implicated in the pathophysiology of ALS. This is because biological macromolecules undergo oxidative damage by reactive oxygen and nitrogen species (ROS/RNS), which leads to organelle dysfunction and neuronal death. Free radical scavengers and other antioxidants are obvious treatment approaches but have had disappointing effects in clinical trials. Advances in ROS/RNS biology explain the failure of such antioxidants and suggest more mechanistic strategies for reducing oxidative stress. ROS/RNS are not merely toxic metabolic waste products. Rather, they are part of complex cellular signaling networks that include ROS/RNS producing enzymes (MPO, NADPH oxidase [NOX], xanthine oxidase), ROS targets (NF-κB mediated inflammatory pathways, inflammasomes), and ROS-metabolizing enzymes (SOD and NRF2-regulated antioxidant pathways). Deregulation of ROS/RNS pathways contributes to other known neurodegenerative disease mechanisms, i.e., microglial activation and neuroinflammation. Thus, it may be advantageous to inhibit ROS/RNS producing enzymes, such as MPO, thereby reducing oxidative stress and inflammation in a physiologic manner. The general relevance of this strategy is supported by preliminary data that suggest inhibiting NOX reduces microglial activation and may have other benefits in an ALS model [7].

### Neuroinflammation

Based on extensive supportive evidence from human ALS and animal models, neuroinflammation is also thought to be an important mediator of neurodegeneration in ALS and other disorders [8, 9]. Microglia, the primary innate immune cells of the brain, are central players that drive neuroinflammation. In disease, resting microglia are activated or transformed into a pathological “M1” phenotype that is characterized by secretion of pro-inflammatory cytokines and ROS/RNS as well as an ameboid morphology and phagocytic properties similar to peripheral macrophages. Modulating microglial activation is a promising and common therapeutic strategy for neurodegenerative diseases, including ALS. However, it is unclear how to determine the optimal molecular target for intervention in this dynamic, in vivo process from studies in disease models.

Positron emission tomography (PET) imaging of translocator protein (TSPO) is a state-of-the-art method for assessing pathological neuroinflammation in human disease states. Because TSPO is highly expressed in activated microglia, signal from TSPO-specific radioligands, such as [¹¹C]-PBR28, is interpreted as a measure of microglial activation. [¹¹C]-PBR28 PET signal is dynamic, responsive to treatment, and potentially predictive of benefit on clinical outcome measures. Thus, [¹¹C]-PBR28 PET is a valuable pharmacodynamic biomarker for anti-inflammatory therapies that can demonstrate central target engagement and provide proof of mechanism of action in human neurodegenerative disorders. Cross-sectional and longitudinal [¹¹C]-PBR28 PET studies in participants with ALS show that [¹¹C]-PBR28 signal is increased in ALS but stable over at least 6 months of disease progression. Corresponding sample size and power calculations suggest [¹¹C]-PBR28 PET is a sensitive biomarker allowing for efficient signal detection in smaller, early phase studies. Several such studies are ongoing to assess investigational treatments in ALS, however none of these treatments has yet demonstrated the ability to decrease [¹¹C]-PBR28 uptake. By contrast, MPO inhibition is the only approach that has demonstrated the ability to decrease [¹¹C]-PBR28 uptake in human neurodegenerative disease, highlighting the therapeutic relevance of this target for ALS.

### Additional Disease Implications

MPO may also play a role in increasingly recognized disease mechanisms mediated by peripheral myeloid cells, including those that migrate into the brain as well as those that remain in the periphery [8], suggesting relevance of MPO as a therapeutic target at both sites. For example, circulating immune cell numbers (particularly neutrophils) appear to correlate with ALS disease progression measured by ALSFRS-R. And, MPO⁺ neutrophils seem to contribute to distal motor axon and muscular pathology in human ALS autopsy tissue and in an ALS animal model.

### Dosage Selection and Justification

The dosage of verdiperstat selected for evaluation in this study is 600 mg BID. This dosage was selected based on cumulative experience, including nonclinical toxicology and safety/tolerability, pharmacokinetic, pharmacodynamic, and preliminary efficacy data from Phase 1 and Phase 2 studies.

Nonclinical toxicology: The program for verdiperstat is comprehensive and supports oral administration in the clinic for chronic treatment. The following studies were included in the toxicology program: single and repeat-dose toxicity in rats and dogs, genotoxicity, reproductive toxicity, phototoxicity, and safety pharmacology. The verdiperstat 600 mg BID dosage is anticipated to produce pharmacokinetic exposures below limits set based on non-clinical toxicology data.

Clinical studies: To date, approximately 250 subjects have received verdiperstat in 4 Phase 1 and 3 Phase 2 studies. In the Phase 1 studies in healthy subjects, treatment with multiple dosages of up to 900 mg BID was generally safe and well tolerated. In the Phase 2 studies in participants with Parkinson’s disease and Multiple System Atrophy (MSA), treatment at dosages of up to 600 mg BID for 8-12 weeks was generally safe and well tolerated. In the Phase 2 studies, the 600 mg BID dosage decreased MPO activity in plasma, providing evidence of peripheral target engagement; reduced TSPO binding on brain PET imaging, providing evidence of central target engagement and proof of mechanism (decreased microglial activation/neuroinflammation); and demonstrated favorable, dose-dependent trends on clinical efficacy measures at 12 weeks in subjects with MSA.

### STUDY DESCRIPTION

### Brief Description

The HEALEY ALS Platform Trial is a perpetual multi-center, multi-regimen clinical trial evaluating the safety and efficacy of verdiperstat for the treatment of ALS.

### Detailed Description

The HEALEY ALS Platform Trial is a perpetual multi-center, multi-regimen clinical trial evaluating the safety and efficacy of investigational products for the treatment of ALS. This trial is designed as a perpetual platform trial. This means that there is a single Master Protocol dictating the conduct of the trial. The HEALEY ALS Platform Trial Master Protocol is registered as NCT04297683.

Once a participant enrolls into the Master Protocol and meets all eligibility criteria, the participant will be eligible to be randomized into any currently enrolling regimen. All participants will have an equal chance of being randomized to any currently enrolling regimen.

If a participant is randomized to Regimen B - Verdiperstat, the participant will complete a screening visit to assess additional Regimen B eligibility criteria. Once Regimen B eligibility criteria are confirmed, participants will complete a baseline assessment and be randomized in a 3:1 ratio to either active Verdiperstat or matching placebo.

Regimen B will enroll by invitation as participants may not choose to enroll in Regimen B. Participants must first enroll into the Master Protocol and be eligible to participate in the Master Protocol before being able to be randomly assigned to Regimen B.

For a list of enrolling sites, please see the HEALEY ALS Platform Trial Master Protocol under NCT04297683 and NCT04436510.

Any additional details and specific information that govern the testing of verdiperstat, and all regimen-specific outcome measures can be found at website ClinicalTrials.gov, wherein all information disclosed at the ClinicalTrials.gov website is incorporated herein in its entirety by reference.

STUDY DESIGN

| | |
|---|---|
| Study Type: | Interventional (Clinical Trial) |
| Estimated Enrolment: | 160 participants |
| Allocation: | Randomized |
| Intervention Model: | Parallel Assignment |
| Masking: | Quadruple (Participant, Care Provider, Investigator, Outcomes Assessor) |
| Primary Purpose: | Treatment |
| Official Title: | HEALEY ALS Platform Trial -Regimen B Verdiperstat |
| Actual Study Start Date: | Jul. 28, 2020 |
| Estimated Primary Completion Date: | October 2021 |
| Estimated Study Completion Date | November 2022 |

ARMS AND INTERVENTIONS

| | |
|---|---|
| Arm | Intervention/Treatment |
| Experimental: Verdiperstat Verdiperstat is administered twice daily p.o. for 24 weeks. | Drug: Verdiperstat Administration: Oral Dose: 600 mg twice daily |
| Placebo Comparator: Matching Placebo Matching placebo is administered twice daily p.o. for 24 weeks. | Drug: Matching Placebo Administration: Oral Dose: two tablets twice daily |

### OUTCOME MEASURES

### Primary Outcome Measures

### 1. Disease Progression [Time Frame: 24 Weeks]

Change in disease severity over time as measured by the ALS Functional Rating Scale-Revised (ALSFRS-R). Each type of function is scored from 4 (normal) to 0 (no ability), with a maximum total score of 48 and a minimum total score of 0. Patients with higher scores have more physical function.

### Secondary Outcome Measures

### 1. Respiratory Function [Time Frame: 24 Weeks]

Change in respiratory function over time as measured by Slow Vital Capacity (SVC).

### 2. Muscle Strength [Time Frame: 24 Weeks]

Change in muscle strength over time as measured isometrically using hand-held dynamometry (HHD).

### 3. Survival [Time Frame: 24 Weeks]

Comparison of rate of occurrence between groups.

### ELIGIBILITY CRITERIA

### Inclusion Criteria

1. Sporadic or familial ALS diagnosed as clinically possible, probable, lab-supported probable, or definite ALS defined by revised El Escorial criteria.
2. Age 18 years or older.
3. Capable of providing informed consent and complying with study procedures, in the SI’s opinion.
4. Time since onset of weakness due to ALS ≤ 36 months at the time of the Master Protocol Screening Visit.
5. Vital Capacity ≥ 50% of predicted capacity for age, height, and sex at the time of the Master Protocol Screening Visit measured by Slow Vital Capacity (SVC), or, if required due to pandemic-related restrictions, Forced Vital Capacity (FVC).
6. Participants must either not take riluzole or be on a stable dose of riluzole for ≥ 30 days prior to the Master Protocol Screening Visit. Riluzole-naive participants are permitted in the study.
7. Participants must either not take edaravone or have completed at least one cycle of edaravone prior to the Master Protocol Screening Visit. Edaravone-naïve participants are permitted in the study.
8. Participants must have the ability to swallow pills and liquids at the time of the Master Protocol Screening Visit and, in the SI’s opinion, have the ability to swallow for the duration of the study.
9. Geographically accessible to the site.

### Exclusion Criteria

1. Clinically significant unstable medical condition (other than ALS) that would pose a risk to the participant, according to SI’s judgment (e.g., cardiovascular instability, systemic infection, untreated thyroid dysfunction, or clinically significant laboratory abnormality or EKG changes). Lab abnormalities include, but are not limited to: Hemoglobin < 10 g/dL, White Blood Cells < 3.0 × 10³/mm³, Neutrophils, Absolute ≤ 1000/mm³, Eosinophilia (absolute eosinophil count of ≥ 500 eosinophils per microliter), low platelet counts (< 150 × 109 per liter), alanine aminotransferase (ALT) or aspartate aminotransferase (AST) greater than 3 times the upper limit of normal (ULN), eGFR < 30 mL/min/1.73 m², thyroid-stimulating hormone (TSH) levels >10 mIU/L or <0.01 mIU/L.
2. Presence of unstable psychiatric disease, cognitive impairment, dementia or substance abuse that would impair ability of the participant to provide informed consent, in the SI’s opinion.
3. Active cancer or history of cancer, except for the following: basal cell carcinoma or successfully treated squamous cell carcinoma of the skin, cervical carcinoma in situ, prostatic carcinoma in situ, or other malignancies curatively treated and with no evidence of disease recurrence for at least 3 years.
4. Use of investigational treatments for ALS (off-label use or active participation in a clinical trial) within 5 half-lives (if known) or 30 days (whichever is longer) prior to the Master Protocol Screening Visit.
5. Exposure at any time to any gene therapies under investigation for the treatment of ALS (off-label use or investigational).
6. If female, breastfeeding, known to be pregnant, planning to become pregnant during the study, or of child-bearing potential and unwilling to use effective contraception for the duration of the trial and for 3 months, or longer as specified in each RSA, after discontinuing study treatment.
7. If male of reproductive capacity, unwilling to use effective contraception for the duration of the trial and for 3 months, or longer as specified in each RSA, after discontinuing study treatment.
8. Anything that would place the participant at increased risk or preclude the participant’s full compliance with or completion of the study, in the SI’s opinion.
9. If a participant is being re-screened, the disqualifying condition has not been resolved, or the mandatory wash-out duration has not occurred.
10. For those participating in the optional CSF collection, contraindication to undergoing a lumbar puncture (LP) in the SI’s opinion. Participants undergoing the LP must not be currently taking anticoagulation medications such as warfarin that would be a contraindication to LP; aspirin and non-steroidal anti-inflammatories are allowed.
11. Participants who are taking strong inhibitors of CYP1A2 (i.e., ciprofloxacin, enoxacin, fluvoxamine, zafirlukast) for chronic/long-term use defined as more than two weeks.
12. Participants who are taking strong inhibitors of CYP3A4 (i.e., conivaptan, itraconazole, ketoconazole, posaconazole, troleandomycin, voriconazole, clarithromycin, diltiazem, idelalisib, nefazodone, and certain antiviral agents [cobicistat, danoprevir, ritonavir, elvitegravir, indinavir, lopinavir, paritaprevir, ombitasavir, dasabuvir, saquinavir, tipranavir, nelfinavir]) for chronic/long-term use defined as more than two weeks. Note: Topical antifungal use is not exclusionary. Participants should not consume large quantities of grapefruit juice (more than 8 oz. per day) on a regular basis.

Sexes eligible for the study: all.

Ages eligible for the study: 18 years or older.

The study does not accept healthy volunteers.

### REFERENCES

Lefkowitz, D.L., and Lefkowitz, S.S. (2008). Microglia and myeloperoxidase: a deadly partnership in neurodegenerative disease. Free Radic Biol Med 45, 726-731.

Pravalika, K., Sarmah, D., Kaur, H., Wanve, M., Saraf, J., Kalia, K., Borah, A., Yavagal, D.R., Dave, K.R., and Bhattacharya, P. (2018). Myeloperoxidase and Neurological Disorder: A Crosstalk. ACS Chem Neurosci 9, 421-430.

Biohaven Pharmaceuticals Inc. Investigator Brochure BHV-3241, Edition 1.o, January 2019.

Singh, A., Kukreti, R., Saso, L., and Kukreti, S. (2019). Oxidative Stress: A Key Modulator in Neurodegenerative Diseases. Molecules 24.

Casas, A.I., Dao, V.T., Daiber, A., Maghzal, G.J., Di Lisa, F., Kaludercic, N., Leach, S., Cuadrado, A., Jaquet, V., Seredenina, T., et al. (2015). Reactive Oxygen-Related Diseases: Therapeutic Targets and Emerging Clinical Indications. Antioxidants & redox signaling 23, 1171-1185.

van der Vliet, A., Eiserich, J.P., Halliwell, B., and Cross, C.E. (1997). Formation of reactive nitrogen species during peroxidase-catalyzed oxidation of nitrite. A potential additional mechanism of nitric oxide-dependent toxicity. The Journal of biological chemistry 272, 7617-7625.

Seredenina, T., Nayernia, Z., Sorce, S., Maghzal, G.J., Filippova, A., Ling, S.C., Basset, O., Plastre, O., Daali, Y., Rushing, E.J., et al. (2016). Evaluation of NADPH oxidases as drug targets in a mouse model of familial amyotrophic lateral sclerosis. Free Radic Biol Med 97, 95-108.

Beers, D.R., and Appel, S.H. (2019). Immune dysregulation in amyotrophic lateral sclerosis: mechanisms and emerging therapies. Lancet Neurol 18, 211-220.

Hammond, T.R., Marsh, S.E., and Stevens, B. (2019). Immune Signaling in Neurodegeneration. Immunity 50, 955-9.

Chew, S., and Atassi, N. (2019). Positron Emission Tomography Molecular Imaging Biomarkers for Amyotrophic Lateral Sclerosis. Front Neurol 10, 135.

Murdock, B.J., Zhou, T., Kashlan, S.R., Little, R.J., Goutman, S.A., and Feldman, E.L. (2017). Correlation of Peripheral Immunity With Rapid Amyotrophic Lateral Sclerosis Progression. JAMA neurology 74, 1446-1454.

Trias, E., King, P.H., Si, Y., Kwon, Y., Varela, V., Ibarburu, S., Kovacs, M., Moura, I.C., Beckman, J. S., Hermine, O., et al. (2018). Mast cells and neutrophils mediate peripheral motor pathway degeneration in ALS. JCI Insight 3.

D0490C00001. A First Time in Man, Single-Centre, Placebo Controlled, Randomized, Double-blind (within dose panel) Study in Healthy Volunteers to Evaluate Safety, Tolerability and Pharmacokinetics after Oral Single Ascending Doses of AZD3241: AstraZeneca; 2007.

D0490C00012. A Single-Centre, Placebo-Controlled, Randomised, Double-Blind Study to Evaluate the Safety, Tolerability and Pharmacokinetics of AZD3241 following Administration of Single Ascending (Part A) and Fractionated Ascending (Part B) Oral Doses to Young Healthy Volunteers: AstraZeneca; 2009.

D0490C00005. A Phase IIa, 12 Week, Multicentre, Double-Blind, Randomized, Placebo Controlled, Parallel-Group Study to Assess the Safety and Tolerability of Oral AZD3241 in Patients with Parkinson’s Disease: AstraZeneca; 2014.

D0490C00002. A Phase I, Single Centre, Double-blind, Randomised, Placebo-controlled, Parallel-group Study to Assess the Safety, Tolerability and Pharmacokinetics of Extended Release Tablets of AZD3241 after Administration of Multiple Ascending Doses in Healthy Male and Female Volunteers including the Effect of Food: AstraZeneca; 2010.

Throughout this application, various publications are referenced by author name and date, or by patent number or patent publication number. The disclosures of these publications are hereby incorporated in their entireties by reference into this application in order to more fully describe the state of the art as known to those skilled therein as of the date of the invention described and claimed herein. However, the citation of a reference herein should not be construed as an acknowledgement that such reference is prior art to the present invention.

Those skilled in the art will recognize, or be able to ascertain using no more than routine experimentation, numerous equivalents to the specific procedures described herein. Such equivalents are considered to be within the scope of this invention and are covered by the following claims. For example, pharmaceutically acceptable salts other than those specifically disclosed in the description and Examples herein can be employed. Furthermore, it is intended that specific items within lists of items, or subset groups of items within larger groups of items, can be combined with other specific items, subset groups of items or larger groups of items whether or not there is a specific disclosure herein identifying such a combination.

What is claimed is:

1. A method for treating amyotrophic lateral sclerosis, comprising administering to a subject in need of such treatment an effective amount of a myeloperoxidase inhibitor.

2. The method of claim 1, wherein the myeloperoxidase inhibitor is 1-(2-isopropoxyethyl)-2-thioxo-1,2,3,5-tetrahydro-pyrrolo[3,2-d]pyrimidin-4-one or a pharmaceutically acceptable salt thereof.

3. The method of claim 1 or 2, wherein the treating comprises slowing progression of amyotrophic lateral sclerosis, reducing intensity of symptoms associated with amyotrophic lateral sclerosis, reducing onset of symptoms associated with amyotrophic lateral sclerosis, reducing weight loss associated with amyotrophic lateral sclerosis, reversing weight loss associated with amyotrophic lateral sclerosis, delaying mortality associated with amyotrophic lateral sclerosis, and combinations thereof.

4. The method of claim 3, wherein the symptoms associated with amyotrophic lateral sclerosis are selected from the group consisting of fine motor function, gross motor function, balbar function, respiratory function, and a combinations thereof.

5. The method of claim 3 or 4, wherein the symptoms associated with amyotrophic lateral sclerosis are selected from the group consisting of walking, speech, eating, swallowing, writing, climbing stairs, cutting food, turning in bed, salivation, dressing, maintaining hygiene, breathing, dyspnea, orthopnea, respiratory insufficiency, and combinations thereof.

6. The method of any one of claims 1-5, wherein the effective amount is about 150 mg or more per day.

7. The method of any one of claims 1-6, wherein the effective amount is about 300 mg or more per day.

8. The method of any one of claims 1-7, wherein the effective amount is about 600 mg or more per day.

9. The method of any one of claims 1-8, wherein the effective amount is about 1200 mg per day.

10. The method of any one of claims 1-9, wherein administering comprises administering a dose equal to about half of the daily dose two times per day.

11. The method of any one of claims 1-10, wherein administering comprises administering a dose equal to about half of a daily dose every 12 hours.

12. The method of any one of claims 1-11, wherein administering comprises administering a dose equal to about one quarter of a daily dose four times per day.

13. The method of any one of claims 1-12, wherein administering comprises administering about 600 mg two times per day.

14. The method of any one of claims 1-13, wherein administering comprises administering about 300 mg four times per day.

15. The method of any one of claims 1-14, wherein the method is carried out for a time period selected from the group consisting of at least about 12 weeks, at least about 24 weeks, at least about 6 months, at least about 1 year, at least about 2 years, at least about 3 years, at least about 4 years, at least about 5 years, at least about 10 years, and until the patient dies.

16. The method of any one of claims 1-15, wherein the method is carried out at least daily for an indefinite amount of time.

17. The method of any one of claims 1-16, further comprising administering one or more other amyotrophic lateral sclerosis treatments simultaneously or concurrently with administering 1-(2-isopropoxyethyl)-2-thioxo-1,2,3,5-tetrahydro-pyrrolo[3,2-d]pyrimidin-4-one or a pharmaceutically acceptable salt thereof.

18. The method of claim 17, wherein the one or more other amyotrophic lateral sclerosis treatment includes riluzole.

19. The method of claim 17 or 18, wherein the one or more other amyotrophic lateral sclerosis treatment includes a sublingual formulation of riluzole.

20. The method of claim 17, wherein the one or more other amyotrophic lateral sclerosis treatment includes a prodrug of riluzole.

21. The method of claim 20, wherein the prodrug of riluzole is troriluzole.

22. The method of any one of claims 1-21, wherein the patient began exhibiting symptoms of amyotrophic lateral sclerosis less than about two years before beginning administering 1-(2-isopropoxyethyl)-2-thioxo-1,2,3,5-tetrahydro-pyrrolo[3,2-d]pyrimidin-4-one or a pharmaceutically acceptable salt thereof.

23. The method of any one of claims 1-22, wherein the patient began exhibiting symptoms of amyotrophic lateral sclerosis at least greater than about two years before beginning administering 1-(2-isopropoxyethyl)-2-thioxo-1,2,3,5-tetrahydro-pyrrolo[3,2-d]pyrimidin-4-one or a pharmaceutically acceptable salt thereof.

24. The method of any one of claims 1-23, wherein the patient exhibits a greater than 20% improvement in ALS Functional Rating Scale, Revised (ALSFRS-R) score when compared to baseline.

25. The method of any one of claims 1-24, wherein the patient exhibits a greater than 30% improvement in ALS Functional Rating Scale, Revised (ALSFRS-R) score when compared to baseline.

26. The method of claim 24 or 25, wherein the improvement is apparent in a time period selected from the group consisting of less than about 9 months, less than about 6 months, less than about 3 months, and less than about 1 month.

27. The method of any one of claims 1-26, wherein administering 1-(2-isopropoxyethyl)-2-thioxo-1,2,3,5-tetrahydro-pyrrolo[3,2-d]pyrimidin-4-one or a pharmaceutically acceptable salt thereof results in slowing of a rate of fine motor function loss in the patient.

28. The method of any one of claims 1-27, further comprising administering a daily dose of greater than an effective amount for a period of time before administering an effective amount of 1-(2-isopropoxyethyl)-2-thioxo-1,2,3,5-tetrahydro-pyrrolo[3,2-d]pyrimidin-4-one or a pharmaceutically acceptable salt thereof.

29. The method of claim 28, wherein the greater than an effective amount is greater than 600 mg.

30. The method of claim 28 or 29, wherein the greater than an effective amount is greater than 900 mg.

31. The method of any one of claims 28-30, wherein the greater than an effective amount is greater than 1200 mg.

32. The method of any one of claims 28-31, wherein the period of time before administering a greater than effective amount is from about 1 weeks to about 12 weeks.

33. The method of any one of claims 28-32, wherein the period of time before administering a greater than effective amount is from about 2 weeks to about 6 weeks.

34. The method of any one of claims 28-33, wherein administering a greater than effective amount of 1-(2-isopropoxyethyl)-2-thioxo-1,2,3,5-tetrahydro-pyrrolo[3,2-d]pyrimidin-4-one or a pharmaceutically acceptable salt thereof is carried out indefinitely.

35. The method of any one of claims 1-34, wherein an effective amount of 1-(2-isopropoxyethyl)-2-thioxo-1,2,3,5-tetrahydro-pyrrolo[3,2-d]pyrimidin-4-one or a pharmaceutically acceptable salt thereof is administered in an initial dose and every administration thereafter.

36. The method of any one of claims 1-35, wherein the effective amount comprises a stable daily dose.

37. The method of claim 36, wherein the stable daily dose comprises about 1200 mg of 1-(2-isopropoxyethyl)-2-thioxo-1,2,3,5-tetrahydro-pyrrolo[3,2-d]pyrimidin-4-one or a pharmaceutically acceptable salt thereof.

38. The method of claim 36 or 37, wherein the stable daily dose comprises 1 to 5 unit doses per day.

39. The method of any one of claims 36-38, wherein each unit dose is a solid unit dose.

40. The method of any one of claims 36-39, wherein administering comprises administering one unit dose two times per day wherein each unit dose is equal to about half of the stable daily dose.

41. The method of any one of claims 36 to 40, wherein administering comprises administering one unit dose once every 12 hours wherein each unit dose is equal to about half of the stable daily dose.

42. The method of any one of claims 36-41, wherein administering comprises administering one unit dose four times per day wherein each unit dose is equal to about one quarter of the stable daily dose.

43. The method of any one of claims 36-42, wherein administering comprises administering two unit doses wherein each unit dose is about 600 mg two times per day.

44. The method of any one of claims 36-43, wherein administering comprises administering four unit doses wherein each unit dose is about 300 mg four times per day.

45. The method of any one of claims 36-44, wherein administering a stable daily dose of 1-(2-isopropoxyethyl)-2-thioxo-1,2,3,5-tetrahydro-pyrrolo[3,2-d]pyrimidin-4-one or a pharmaceutically acceptable salt thereof is carried out for at least about 12 weeks.

46. The method of any one of claims 36-45, wherein administering a stable daily dose of 1-(2-isopropoxyethyl)-2-thioxo-1,2,3,5-tetrahydro-pyrrolo[3,2-d]pyrimidin-4-one or a pharmaceutically acceptable salt thereof is carried out for an indefinite amount of time.

47. The method of any one of claims 36-46, wherein the stable daily dose is consistent throughout a treatment regimen.

48. The method of any one of claims 36-47, wherein an initial daily dose is equal to each daily dose thereafter.

49. The method of any one of claims 36-48, wherein there is no titration before administering the stable daily dose.

50. The method of any one of claims 1-49, further comprising monitoring the patient.

51. The method of any one of claims 1-50, further comprising monitoring the patient for neutropenia.

52. The method of any one of claims 1-51, further comprising monitoring ALSFRS-R score for the patient.

53. The method of any one of claims 1-52, further comprising monitoring the patients fine motor function, gross motor function, bulbar function, respiratory function, and combinations thereof.

54. The method of any one of claims 1-53, further comprising monitoring behaviors selected from the group consisting of swallowing, handwriting, speech, ability to walk, ability to climb stairs, ability to dress, ability to maintain hygiene, and combinations thereof.

55. The method of any one of claims 1-54, further comprising scheduling a doctor visit every 6 months for at least 12 months.

56. The method of any one of claims 1-55, wherein the patient is predisposed to amyotrophic lateral sclerosis and is not exhibiting symptoms of amyotrophic lateral sclerosis.

57. The method of any one of claims 1-56, further comprising administering 1-(2-isopropoxyethyl)-2-thioxo-1,2,3,5-tetrahydro-pyrrolo[3,2-d]pyrimidin-4-one or a pharmaceutically acceptable salt thereof to family members of the patient.

58. The method of any one of claims 1-56, wherein treating comprises administering 1-(2-isopropoxyethyl)-2-thioxo-1,2,3,5-tetrahydro-pyrrolo[3,2-d]pyrimidin-4-one or a pharmaceutically acceptable salt thereof to a patient not exhibiting symptoms of amyotrophic lateral sclerosis.

59. The method of any one of claims 1-58, wherein treating comprises administering 1-(2-isopropoxyethyl)-2-thioxo-1,2,3,5-tetrahydro-pyrrolo[3,2-d]pyrimidin-4-one or a pharmaceutically acceptable salt thereof to a patient that is predisposed to amyotrophic lateral sclerosis.
